(19) United States
(12) Patent Application Publication
(10) Pub. No.: US 2010/0004595 A1
(43) Pub. Date: Jan. 7, 2010

(54) BALLOON CATHETER SYSTEMS FOR TREATING UTERINE DISORDERS HAVING FLUID LINE DE-GASSING ASSEMBLIES AND METHODS THEREFOR

(75) Inventors: Steven Nguyen, North Brunswick, NJ (US); David Szabo, Doylestown, PA (US)

Correspondence Address: Doherty IP Law Group LLC, 37 Belvidere Ave, Washington, NJ, 07882, US

(73) Assignee: Ethicon, Inc., Somerville, NJ (US)

(21) Appl. No.: 12/165,675

(22) Filed: Jul. 1, 2008

(51) Int. Cl. A61M 25/10

(52) U.S. Cl. 604/99.04

(57) ABSTRACT

A system for treating uterine disorders includes a catheter with a cannula having a proximal end and a distal end, and a degassing system in communication with the distal end of the cannula. The degassing system has a fluid insertion path having a first check valve and a gas filter, and a fluid extraction path that is separate from the fluid insertion path and includes a second check valve. The catheter may include an inflatable balloon secured to the distal end of the cannula with the degassing system in communication with the inflatable balloon. In one embodiment, a heating assembly is disposed inside the inflatable balloon for heating the fluid introduced into the balloon.

### BACKGROUND OF THE INVENTION

1. Field of the Invention

The present application is generally related to treating uterine disorders and is more specifically related to balloon catheter systems and methods for treating uterine disorders.

2. Description of the Related Art

Excessive or abnormal uterine bleeding in premenopausal females, commonly referred to as menorrhagia, has been a leading cause of about 30% of the hysterectomies performed in the United States. Women afflicted with menorrhagia typically lose 10 to 25 times the normal amount of blood during their menstrual cycle and often contend with iron deficiencies, pain, fatigue, and the inability to participate in daily activities. While hysterectomies are effective, less invasive outpatient procedures have been introduced that preserve the uterus and reduce recovery time. One procedure, commonly referred to as endometrial ablation, involves inserting a balloon catheter filled with a heated fluid into the uterus. In one embodiment of a system sold under the trademark THERMACHOICE III by Johnson & Johnson of New Brunswick, N.J., a balloon catheter is inserted into a uterus, and inflated with a 5% dextrose solution. After the balloon is inflated with the solution, the solution is heated to a predetermined temperature for a period of time that coagulates, ablates, necroses, or destroys the endometrium layer of the uterus. After the procedure is completed, the solution is withdrawn from the balloon and the balloon is removed from the uterus. The uterine lining will then shed over a 7-10 day period.

During endometrial ablation procedures, the solution or fluid inside the balloon must be heated to a predetermined temperature level. Temperature fluctuations and gradients along the surface of the balloon may cause uneven tissue ablation resulting in a less than optimal outcome. In many instances, balloon surface temperature fluctuations and gradients are the result of the fluid not mixing fully within the balloon. When the fluid is not completely mixed, the fluid temperature is subject to convection currents within the balloon. While cooler fluid moves toward the bottom of the balloon, the warmer, less dense fluid rises. When the fluid within the balloon is subject to such convection currents during heating, considerable temperature fluctuations along the surface of the balloon may result.

Some balloon catheters circulate fluid by means of separate inlet and outlet passages that connect the balloon with an external heating element. Heat is circulated from the external heating element through the inlet passage into the balloon. Then, the fluid from the balloon is returned to the external heating element through the outlet passage. Such a balloon catheter design requires the hot fluid to pass through the vagina and the opening of the cervix, which may cause physical discomfort or possible tissue damage as heat is conducted through the balloon catheter walls. Since the hot fluid must travel a significant distance between the external heating element and the balloon surface being heated, efficient control over the temperature of the balloon surface is difficult.

Other known heated balloon catheters circulate fluid via a pair of one way valves mounted within a housing located at the end of a fluid delivery tube. The housing is surrounded by an inflatable member, such as a balloon. The first valve permits fluid flow from the housing into the balloon, and the second valve permits flow from the balloon into the housing. The valves respond to alternating pressure differentials between the balloon and the housing created by an external bellows or piston which causes pulses of fluid to move up and down the fluid delivery tube. Such a configuration requires circulating hot fluid from the balloon into the fluid delivery tube, creating a risk of causing discomfort to the patient or vaginal tissue damage.

Mechanical circulation or agitation of fluid within the balloon has been known to improve temperature consistency over the surface of the balloon. For example, commonly assigned U.S. Pat. No. 5,954,714, the disclosure of which is hereby incorporated by reference herein, teaches a device for endometrial ablation procedures including a balloon having an internal heater for heating a fluid to a desired temperature. A rotary impeller is positioned distally of the heater for causing the fluid inside the balloon to move around the balloon.

In one embodiment of commonly assigned U.S. patent application Ser. No. 12/134,265, filed Jun. 6, 2008, the disclosure of which is hereby incorporated by reference herein, a balloon catheter system includes an elongated heating tube and an impeller for circulating fluid within the elongated heating tube. The elongated heating tube includes inlet openings at one end of the tube and an outlet opening at the distal end of the tube. As the impeller rotates, fluid is drawn into the inlet openings, moves along the length of the heating tube, and is discharged from the outlet opening at the distal end of the tube. Positioning the impeller inside the heating tube ensures positive communication between the fluid and the heating tube, thereby improving heating consistency throughout the inside of the balloon.

In many instances, air or gas remains trapped inside the balloon with the fluid. As a result, air or gas pockets may develop inside the balloon during an endometrial ablation procedure. The air or gas pockets interrupt thermal consistency throughout the balloon, which may reduce the efficacy of the endometrial ablation procedure. There have been a number of attempts seeking to remove air from inside fluid-filled balloons during endometrial ablation procedures. Most of these attempts involve a series of manual steps whereby a syringe is first used to remove any air present in the balloon, and is then used to fill the balloon with a fluid.

In spite of the above advances, there remains a need for balloon catheter systems and methods that more effectively and efficiently prime balloons by removing air or gas pockets that may form inside balloons, that more effectively and efficiently introduce fluid into balloons, that more accurately and efficiently heat the fluid inside the balloons, that more efficiently monitor and control fluid pressure inside the balloons, that more efficiently circulate fluid throughout the balloons, and that more efficiently transfer heat from a heating element to fluid so as to provide more uniform heating of the outer surface of the balloons.

### SUMMARY OF THE INVENTION

As used herein, the terminology “menorrhagia” means a condition of excessive menstrual bleeding in women; “thermal coagulation” means the application of heat to tissue in an amount sufficient to destroy the tissue; “necrosis” means the death of cells in tissue; and “endometrium” is the mucous membrane lining of the inner surface of the uterus that grows during each menstrual cycle and is shed in menstrual blood.

In one embodiment, a system for treating uterine disorders includes a catheter having a cannula with a proximal end and a distal end, and a degassing system in communication with the distal end of the cannula, the degassing system including a fluid insertion path having a first check valve and a gas filter, and a fluid extraction path separate from the fluid insertion path and including a second check valve. The system may include an inflatable balloon secured to the distal end of the cannula, whereby the degassing system is in communication with the inflatable balloon. The system may also include a heating assembly disposed inside the inflatable balloon.

In one embodiment, the present invention discloses a balloon catheter system including a degassing system having paired one-way check valves allowing for bidirectional flow of fluid, and an air filter to remove air or gas from the fluid as the fluid passes through the degassing system. The filter preferably exhausts the air or gas removed from the fluid to the atmosphere. In one embodiment, the balloon catheter system preferably includes a vent hole located at the distal end of the catheter that is disposed inside the balloon. The vent hole is preferably adapted to allow fluid to be introduced into the balloon. The vent hole also preferably allows air or gas present in the balloon to be extracted from the balloon through the vent hole. In one embodiment, when the balloon is at least partially filled with fluid, the vent hole allows any gas or air mixed in with the fluid to be extracted from the balloon. Thus, the vent hole may be used for introducing fluid into the balloon and extracting air/gas and/or fluid from the balloon.

In one embodiment, the system includes a pump, such as a peristaltic pump, that is automatically controlled by a system controller for priming the inflatable balloon with a heatable fluid. The pump may also operate during therapy, such as during an endometrial ablation procedure. In one embodiment, the priming of the balloon and the steps of the therapy are performed automatically to insure that the steps of the procedure are consistently and repeatedly performed. It is contemplated that any type of well-known pump may be used for priming the balloon catheter and during therapy. In one embodiment, at the option of an operator, the automatic mode may be overridden and one or more of the priming and/or therapy steps may be performed manually.

In one embodiment, the degassing system includes a first one-way check valve and an air filter that is located downstream of the first one-way check valve. The first one-way check valve and the air filter are preferably in communication with one another via a fluid insertion path. The degassing system also includes a second one-way check valve that is not in communication with the first one-way check valve. When one of the check valves is open, the other check valve is desirably closed. The air filter is primarily designed for removing air and/or gas from the fluid as the fluid and air/gas mixture travels through the fluid insertion path toward the inflatable balloon.

Fluid movement through the degassing system may be driven by using a syringe or a pump, such as a peristaltic pump. During a priming procedure, the system may extract all of the fluid and gas from the inflatable balloon to collapse the balloon, whereby the balloon closely conforms to the outer surface of the distal end of the catheter. When the balloon is collapsed, the distal end of the catheter may be inserted into the uterus. After the distal end of the catheter is inserted into the uterus, the inflatable balloon may be filled with the fluid to begin the endometrial ablation procedure.

In one embodiment, a system for treating uterine disorders includes a balloon catheter having a cannula with a proximal end and a distal end, an inflatable balloon secured to the distal end of the cannula, a heating assembly disposed inside the inflatable balloon, and a degassing system in communication with the inflatable balloon. The degassing system includes a fluid insertion path having a first check valve and a gas filter for removing gas (e.g. air) from the fluid, and a fluid extraction path separate from the fluid insertion path that includes a second check valve. In one embodiment, the gas filter is located distally from the first check valve and is adapted to remove any gas or air present in the fluid flowing through the fluid insertion path. As used herein, the terms “gas” and “air” are used interchangeably to describe any gaseous, non-liquid substance that may be present inside an inflatable balloon. As described herein, the degassing system of the present invention is adapted to remove any gas or air disposed inside balloon to insure that no air pockets are disposed inside the balloons.

In one embodiment, the first check valve allows the fluid to flow in a first direction and the second check valve allows the fluid to flow in a second direction that is opposite the first direction. In one embodiment, the first check valve preferably allows the fluid to flow in a distal direction toward the inflatable balloon and the second check valve preferably allows the fluid to flow in a proximal direction, which is away from the inflatable balloon.

In one embodiment, the system includes a system controller in communication with the balloon catheter for controlling operation of the balloon catheter. The system controller may include one or more automatic priming subroutines programmed therein for filling the inflatable balloon with a fluid. The automatic priming subroutines preferably have at least one fluid insertion phase and at least one fluid extraction phase. In highly preferred embodiments, the fluid insertion and extraction phases are continuously repeated during a balloon priming operation until the balloon is completely filled with fluid and no gas or air is present in the balloon. In one embodiment, during the fluid insertion phase, the first check valve is preferably open and the second check valve is preferably closed. In one embodiment, during the at least one fluid extraction phase, the first check valve is closed and the second check valve is open.

In one embodiment, the fluid insertion path and the fluid extraction path are in communication with the inside of the inflatable balloon. The distal end of the cannula desirably includes a vent hole or vent opening disposed inside the inflatable balloon. The vent hole is desirably surrounded by and located adjacent a proximal end of the inflatable balloon, whereby the fluid insertion and fluid extraction paths are in communication with the vent hole. In one embodiment, during a priming operation, the balloon at the distal end of the catheter is pointed downward so that the vent hole is located at the highest point of the balloon. Positioning the vent hole at the highest point during priming facilitates the removal of air first and fluid second to achieve a good priming result whereby the balloon is filled with fluid and all air or gas has been removed from inside the balloon. In one embodiment, the vent hole for extracting the air or gas from the balloon may be separate from a conduit used for introducing fluid into the balloon and extracting fluid from the balloon.

In one embodiment, a system for treating uterine disorders includes a balloon catheter having a handle, and a cannula extending from a distal end of the handle, an inflatable balloon secured to a distal end of the cannula, a heating assembly disposed inside the inflatable balloon, a fluid agitator disposed inside the inflatable balloon for circulating fluid, and a fluid degassing system disposed within the handle and being in communication with the inflatable balloon. The fluid degassing system desirably includes a fluid insertion path having a first check valve and a gas filter, and a fluid extraction path separate from the fluid insertion path and having a second check valve. The first check valve preferably allows fluid to flow in a distal direction toward the inflatable balloon and the second check valve allows fluid to flow in a proximal direction away from the inflatable balloon.

In one embodiment, the system desirably includes a system controller in communication with the balloon catheter for automatically controlling operation of the balloon catheter, or for providing instructions to an operator regarding the status of the procedure and the steps that must be taken to complete the procedure. In one embodiment, the system controller includes a priming subroutine for filling the inflatable balloon with a fluid having at least one fluid insertion phase and at least one fluid extraction phase. In one embodiment, the first check valve is open and the second check valve is closed during each fluid insertion phase, and the first check valve is closed and the second check valve is open during each fluid extraction phase.

In one embodiment, the heating assembly includes an elongated heating tube having a proximal end, a distal end, and an outer wall extending between the proximal and distal ends, at least one fluid inlet extending through the outer wall, and a fluid outlet located at the distal end of the elongated heating tube, whereby the fluid agitator is a rotatable impeller disposed inside the elongated heating tube for drawing fluid through the at least one fluid inlet and into the elongated heating tube for heating fluid inside the balloon. The at least one fluid inlet is preferably located adjacent the proximal end of the elongated heating tube, and the rotatable impeller is adapted to discharge the fluid through the fluid outlet located at the distal end of the elongated heating tube so as to circulate the fluid throughout the inflatable balloon.

In one embodiment, a system for treating uterine disorders includes a balloon catheter having a handle, and a cannula extending from a distal end of the handle, an inflatable balloon secured to a distal end of the cannula and being adapted to receive fluid, a heating assembly disposed inside the inflatable balloon for heating the fluid, and a rotatable impeller disposed inside the inflatable balloon and adjacent the heating assembly for circulating the fluid through the inside of the inflatable balloon. The system preferably includes a fluid degassing system disposed within the handle and being in communication with the inside of the inflatable balloon, the fluid degassing system including a fluid insertion path having a first check valve and a gas filter, and a fluid extraction path separate from the fluid insertion path and having a second check valve.

In one embodiment, the heating assembly includes an elongated heating tube having a proximal end, a distal end, and an outer wall extending between the proximal and distal ends, at least one fluid inlet extending through the outer wall of the heating tube, and a fluid outlet located at the distal end of the elongated heating tube, whereby the rotatable impeller is disposed inside the elongated heating tube for drawing fluid through the at least one fluid inlet and into the elongated heating tube for heating fluid inside the balloon. The distal end of the cannula preferably includes a vent hole and the inflatable balloon has a proximal end that overlies the vent hole so that the vent hole is located inside the balloon and adjacent the proximal end of the inflatable balloon. The fluid insertion and the fluid extraction paths are preferably in communication with the vent hole for introducing fluid into the balloon and extracting any air or gas present in the balloon.

In one embodiment, the balloon catheter is primed by pointing the inflatable balloon located at the distal end of the catheter down toward the floor. Although the present invention is not limited by any particular theory of operation, it is believed that pointing the balloon downward will result in any gas or air in the balloon moving to the highest point inside the balloon. The gas or air at the highest point in the balloon may then be removed using the vent hole, which is co-located at the highest point in the balloon. A fluid source containing a fluid such as D₅W solution is preferably coupled with the balloon catheter for supplying the fluid to the balloon. In one embodiment, the fluid source may include a fluid tube for supplying the fluid to the inflatable balloon. The fluid tube may be placed in contact with a pump such as a peristaltic pump for advancing the fluid through the fluid tube and into the balloon. In one embodiment, the fluid tube is placed in contact with the rollers of a peristaltic pump. The fluid is directed through the fluid tube and into the hand piece of the balloon catheter where it flows through the fluid insertion path of the degassing system. As the fluid flows through the fluid insertion path, the second one-way check valve is closed. The fluid continues past the first one-way check valve and through the air filter, which removes any gas or air present in the fluid. Once the fluid passes through the first one-way check valve and the air filter, it is directed into the balloon through a vent hole located at the distal end of the catheter. Any gas or fluid present in the balloon may be removed from the inflatable balloon by running the pump in the reverse direction.

As the distal end of the balloon catheter remains pointed at the ground, any gas or air remaining in the balloon is extracted through the vent hole that is located at the top of the balloon. In one embodiment, air from the balloon is either returned through the fluid extraction path to the fluid source for being trapped therein, or is removed from the system when it is pushed through the air filter during the next filling cycle. As the fluid is extracted through the fluid extraction path, the first one-way check valve of the fluid insertion path is closed. During fluid filling and extraction, a pressure line in communication with the fluid tube enables the system controller to monitor and/or control the pressure level of the fluid within the system.

In one embodiment, a manual line may also be incorporated into the system for conducting manual or emergency operations. In one embodiment, the manual line includes an inlet that is accessible at the proximal end of the handle of the balloon catheter, whereby a distal end of the syringe may be coupled with the manual line inlet opening. The balloon catheter includes a trumpet valve that may be engaged so that the fluid may be manually inserted into the inflatable balloon or manually removed from the inflatable balloon using the syringe. The manual line enables fluid to be evacuated via the fluid extraction path of the degassing system in an emergency situation or a manual override situation. The manual line also enables users to operate the device in a manual mode. In the manual mode, the fluid travels through the degassing system as it would during the automated mode outlined above.

Although the present invention is not limited by any particular theory of operation, it is believed that providing a degassing system including paired one-way check valves and an air filter assembly in communication with one of the check valves provides a unique arrangement to control the fluid flow path between a fluid source and an inflatable balloon. The specific arrangement of the opposing check valves enables the fluid to flow into the inflatable balloon while removing any gas or air present in the fluid. The fluid insertion path and the fluid extraction path provide two separate and distinct fluid flow paths through the balloon catheter. Separating the fluid extraction path from the fluid insertion path enables the fluid to be extracted from the balloon without passing through the first check valve and the air filter of the fluid insertion path. This situation exists for both automated and emergency/manual modes of operation. The above configuration of the degassing system protects the membrane of the filter from undesired stress during fluid extraction. In one embodiment, there is only one single fluid inlet tube that leads to the degas system and only one single outlet tube at the discharge end of the degas system, whereby only the degas system has two distinct fluid paths (e.g. a fluid insertion path and a fluid extraction path). This preferred design is compact, minimizes the size of the catheter, and eliminates the need for another tube.

In one embodiment of the present invention, a system for treating uterine disorders, such as a system for conducting endometrial ablation procedures, includes a balloon catheter having a cannula with a proximal end and a distal end. The system includes an inflatable balloon secured over the distal end of the cannula, a heating assembly coupled with the distal end of the cannula and disposed inside the inflatable balloon, and an impeller disposed inside the heating assembly. The balloon catheter may include a handle assembly secured to the proximal end of the cannula. The handle assembly may include a fluid fill port for introducing fluid into the inflatable balloon and at least one element (e.g. a fluid fill valve) for controlling operation of the balloon catheter. In one embodiment, fluid may be introduced into the inflatable balloon automatically using a system controller coupled with the balloon catheter.

In one embodiment, the heating assembly includes an elongated tube having an outer wall, at least one fluid inlet extending through the outer wall, and a fluid outlet located at a distal end of the elongated tube. The elongated tube may be an elongated heating tube having a heating film overlying the outer wall of the elongated tube for generating heat. A heating film may also cover an inner surface area of the heating tube. In one embodiment, the total area of the at least one fluid inlet is at least equal to the total area of the fluid outlet. In one embodiment, the at least one fluid inlet includes a plurality of fluid inlets. The one or more fluid inlets are preferably located at the proximal end of the heating tube so that the fluid passing through the inlet(s) is positively directed to engage the heating tube as it moves along the length of the heating tube.

The impeller is preferably rotatable for drawing fluid through the at least one fluid inlet and into the heating assembly for heating the fluid. As the fluid passes by the heating assembly, the heating assembly preferably transfers heat to the fluid via convection. The rotatable impeller is adapted to discharge the fluid through the fluid outlet located at the distal end of the elongated tube so as to circulate the fluid throughout the inflatable balloon. In one embodiment, a balloon catheter system includes the degassing system disclosed herein; however, no heater is disposed inside the balloon. In this particular embodiment, the fluid may be heated outside the balloon and then introduced into the balloon.

In one embodiment, the cannula includes a lumen extending between the proximal and distal ends thereof for introducing a fluid into the inflatable balloon. A pressure monitor may be in communication with the lumen and/or the fluid for monitoring fluid pressure inside the inflatable balloon. The cannula may also include an impeller drive shaft extending therethrough that is coupled with the impeller for rotating the impeller. The drive shaft preferably has a distal end that extends beyond a distal end of the impeller and a protective cap may cover the distal end of the drive shaft for spacing the distal end of the drive shaft and the impeller from the inflatable balloon. The spacing provided by the cap may prevent the balloon from becoming damaged by contacting the rotating drive shaft or the rotating impeller. In one embodiment, the protective cap is insertable into an opening at the distal end of the elongated tube. The protective cap may be insertable into the fluid outlet located at the distal end of the elongated heating tube. The protective cap preferably has one or more openings extending therethrough for enabling fluid to pass by the cap when the cap is secured in place.

In one embodiment, the cannula may also have one or more conductive leads extending therethrough. The conductive leads preferably interconnect one or more of the elements at the distal end of the balloon catheter with the system controller. In one embodiment, the conductive leads may provide power for one or more components of the heating assembly disposed at the distal end of the balloon catheter.

The system may also include a controller for controlling operation of the system. The system controller is preferably used for controlling an endometrial ablation procedure. In one preferred embodiment, the system controller includes a microprocessor for running endometrial ablation routines with a pressure monitoring subroutine for monitoring and controlling the pressure level of the fluid within the balloon, a temperature monitoring subroutine for monitoring and controlling the temperature of the fluid within the balloon, and a timer subroutine for monitoring and controlling how long the endometrial layer of the uterus is exposed to the heated fluid. In a highly preferred embodiment, the system controller automatically performs one or more of the steps of an endometrial ablation procedure.

In one embodiment, once a balloon catheter is positioned within a uterine cavity, fluid is introduced into the inflatable balloon. The fluid is heated, preferably by a heating tube, and circulated within the uterine cavity to heat the lining of the cavity to sufficiently damage the endometrial lining. The heater tube desirably has one or more films coated over the outer diameter of the tube that are adapted to generate heat. An impeller is located along the inner diameter of the heater tube to circulate the fluid. The arrangement of the impeller relative to the heater tube positively ensures that the circulated fluid will pass by the inner diameter surface of the heater tube, which allows the fluid to more effectively absorb heat for reducing the heater temperature set point to heat the fluid to a certain temperature in comparison to the arrangement of having an agitator at the distal end of the heater. Moreover, as a result of fluid being positively moved through the heater, the fluid within the balloon is more efficiently heated and circulated, thereby resulting in a more consistent balloon surface temperature.

In one embodiment of the present invention, a balloon catheter has an impeller located along the inner diameter of a heating assembly, such as a heating assembly having an elongated heating tube. Although the present invention is not limited by any particular theory of operation, it is believed that the arrangement of the impeller relative to the heating assembly improves overall fluid circulation inside the balloon, which improves thermal transfer from the heater to the fluid, and which results in uniform temperature distribution around the outer surface of the balloon. The more uniform temperatures around the outer surface of the balloon promote more uniform treatment of the uterine tissue. In addition, the improved heat transfer between the heating assembly and the fluid results in a reduction in the amount of energy required to heat the fluid. Moreover, better heat transfer enables the system to have a reduced temperature set point while still achieving an appropriate temperature at the outer surface of the balloon.

In one embodiment, the degassing systems disclosed herein may be incorporated into any type of medical device having a catheter. The degassing system of the present invention may be used in any medical device or medical system in which it is desirable to remove gas and/or air from a fluid.

These and other preferred embodiments of the present invention will be described in more detail below.

### BRIEF DESCRIPTION OF THE DRAWING

FIG. 1 shows a perspective view of a system used for endometrial ablation procedures including a system controller, a balloon catheter, a cartridge for connecting the balloon catheter to the system controller, a fluid source, and a fluid source holder, in accordance with one embodiment of the present invention.

FIG. 2 shows another perspective view of the system shown in FIG. 1, including a syringe.

FIG. 3 shows a perspective view of the balloon catheter and the cartridge shown in FIGS. 1 and 2.

FIG. 4 shows a front view of the cartridge shown in FIGS. 1-3.

FIG. 5 shows a front view of the system controller shown in FIGS. 1 and 2.

FIG. 6 shows a cartridge connection port on the front face of the system controller shown in FIG. 5.

FIG. 7 shows the balloon catheter and the cartridge of FIG. 3 connected with the system controller of FIG. 5.

FIG. 8 shows a schematic view of a degassing system for a balloon catheter system, in accordance with one embodiment of the present invention.

FIG. 9 shows a cross-sectional view of the handle section of the balloon catheter shown in FIG. 3.

FIG. 10 shows a perspective view of a degassing system for a balloon catheter system, in accordance with one embodiment of the present invention.

FIG. 11A shows a perspective view of a valve and filter subassembly of the degassing system of FIG. 10.

FIG. 11B shows an exploded view of the valve and filter subassembly of FIG. 11A.

FIG. 12A shows a side view of a distal end of the balloon catheter shown in FIGS. 1-3.

FIG. 12B shows an expanded side view of a portion of the distal end of the balloon catheter shown in FIG. 12A.

FIG. 13A shows a side elevational view of the balloon catheter shown in FIG. 3.

FIG. 13B shows a side view of a distal end of the balloon catheter shown in FIG. 13A.

FIG. 14A shows a side view of a cannula and a heating assembly provided at the distal end of the balloon catheter shown in FIG. 13A.

FIG. 14B shows a cross-sectional view of the cannula and the heating assembly provided at the distal end of the balloon catheter shown in FIG. 14A.

FIG. 15A shows a side elevational view of the heater assembly at the distal end of the balloon catheter shown in FIG. 14A.

FIG. 15B shows a cross-sectional view of the heater assembly shown in FIG. 15A.

FIG. 16 shows a schematic view of an elongated heating tube of a heater assembly, in accordance with one embodiment of the present invention.

FIG. 17 shows a perspective view of an impeller disposable inside the elongated heating tube of FIG. 16.

FIGS. 18A-18C show the steps of an endometrial ablation procedure using the system shown in FIGS. 1-17, in accordance with one embodiment of the present invention.

FIG. 19 shows the path of fluid circulating through the distal end of the balloon catheter of FIG. 3 during one stage of an endometrial ablation procedure, in one embodiment of the present invention.

FIG. 20 shows a visual display provided on a front face of the system controller shown in FIGS. 1-2.

### DETAILED DESCRIPTION

A successful endometrial ablation procedure requires controlling the temperature of the fluid within the balloon and the temperature of the outer surface of the balloon. Temperature fluctuations and gradients along the outer surface of the balloon may be caused by the presence of gas or air pockets inside the balloon, which adversely affects physician control over the endometrial ablation procedure. The systems and methods of the present invention remove gas or air from fluid introduced into the inflatable balloons and remove any gas or air present inside the inflatable balloon, thereby improving temperature consistency along the outer surface of the balloon and the overall efficacy of the endometrial ablation procedure.

In one embodiment, the present invention discloses a system including a balloon catheter used to treat uterine disorders in women, such as menorrhagia, by inserting the balloon catheter into the patient's uterus and inflating the balloon with the fluid, such as a saline or an aqueous sugar solution. After the balloon is inflated with the fluid, the fluid is heated to a predetermined temperature (e.g. 81° C.) for a period of time that coagulates, ablates, necroses, or destroys the endometrium. Utilization of the balloon catheter system of the present invention effectively curtails the excessive uterine bleeding associated with menorrhagia without requiring surgical removal of the uterus. Although a specific temperature is set forth above, other temperatures may be used and still fall within the scope of the present invention.

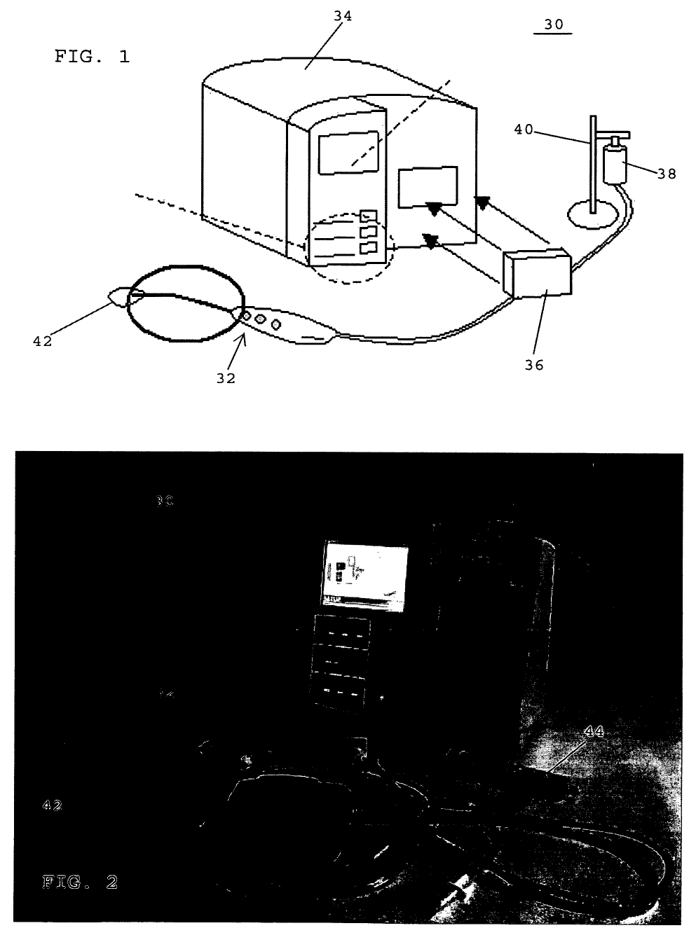

Referring to FIG. 1, in one embodiment of the present invention, a system 30 used for endometrial ablation procedures includes a balloon catheter 32 insertable into a uterine cavity, a system controller 34, a cartridge 36 for connecting the balloon catheter 32 with the system controller 34, a fluid source 38 for supplying fluid to the balloon catheter 32, and a holder 40 for the fluid source. The fluid source 38 is adapted to hold a fluid, such as a D₅W solution or a saline solution, which is introduced into an inflatable balloon 42 located at the distal end of the balloon catheter 32 and heated inside the balloon during the endometrial ablation procedure. The fluid in the fluid source 38 is preferably introduced into the balloon 42 using various techniques well-known to those skilled in the art. In one embodiment, the fluid may be introduced into the balloon 42 manually, such as by using a syringe, or automatically by the system controller via one or more conduits or tubes coupled with the balloon catheter 32.

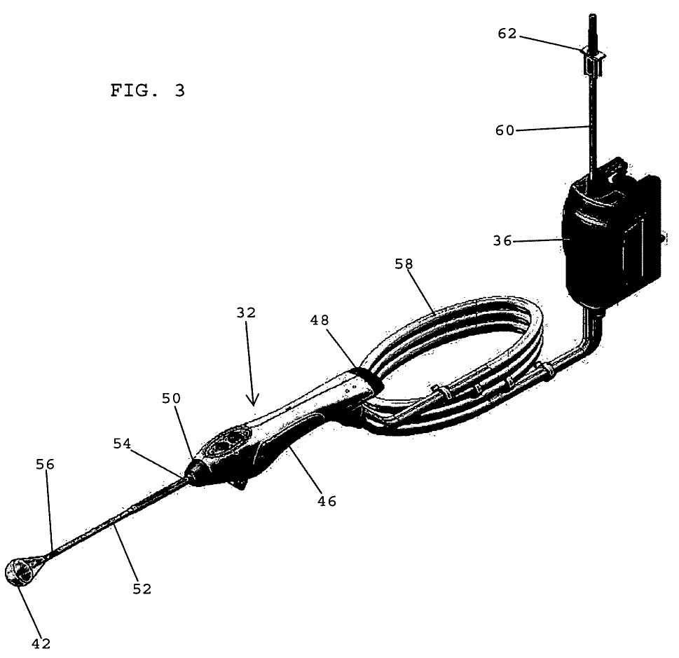

Referring to FIG. 2, in one embodiment, the system 30 preferably includes a syringe 44 that may be used to manually introduce a fluid into the inflatable balloon 42 at a distal end of the catheter 32. The syringe 44 may also be used to remove fluid or gas present inside the balloon 42. In one embodiment, the syringe 44 may be used for emergency evacuation of the fluid from the balloon catheter.

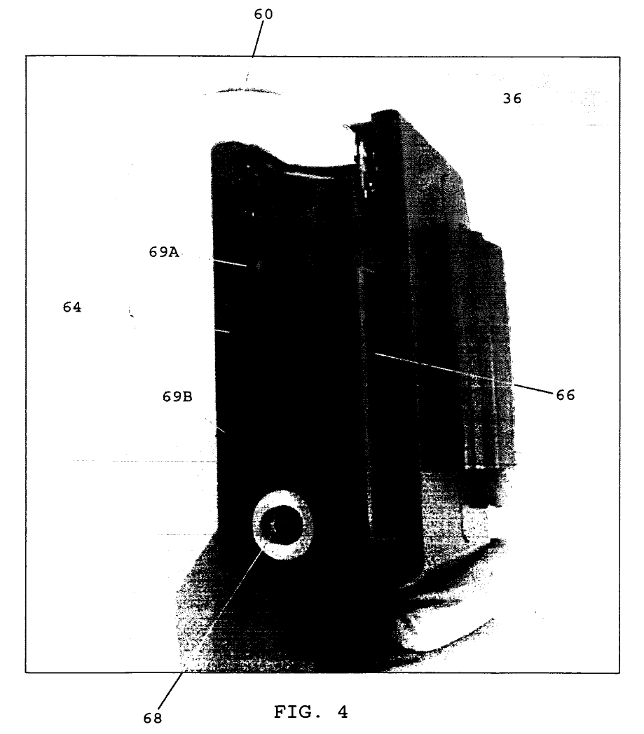

Referring FIG. 3, in one embodiment, the balloon catheter 32 includes a handle 46 having a proximal end 48 and a distal end 50. The balloon catheter 32 includes a flexible cannula 52 extending from the proximal end 50 of the handle 46. The flexible cannula 52 includes a proximal end 54 connected to the distal end 50 of the handle 46 and a distal end 56 remote therefrom. The inflatable balloon 42 extends beyond the distal end 56 of the cannula 52. The balloon catheter 32 also includes a heater (not shown) disposed inside the inflatable balloon for heating fluid introduced into the inflatable balloon. An impeller (not shown) is also disposed inside the balloon 42 for circulating fluid throughout the balloon. In one embodiment, the heating element is a tubular heating element and the rotatable impeller is disposed inside the tubular heating element as disclosed in commonly assigned U.S. patent application Ser. No. 12/134,265, filed Jun. 6, 2008, the disclosure of which is hereby incorporated by reference herein.

In certain preferred embodiments of the present invention, the inflatable balloon 42 is made of latex, silicone, or other elastomeric materials. In one embodiment, the inflatable balloon is approximately 3-5 centimeters in length when inflated by fluid. The inflatable balloon is desirably capable of filling the uterine cavity and exerting pressure against the endometrial layer. The inflatable balloon is desirably capable of withstanding high temperatures without rupturing, and preferably has good heat transfer characteristics to provide efficient heat transfer from the heating assembly to the uterine tissue. The inflation medium or heating fluid is preferably a sterile non-toxic fluid. In one embodiment, the fluid is a solution of five percent (5%) dextrose in water.

The system also preferably includes the cartridge 36 that is coupled with the balloon catheter 32 via one or more cables and/or tubes 58 extending between the cartridge 36 and the balloon catheter 32. The one or more cables 58 are preferably adapted for providing fluid, power and/or control signals to and from the balloon catheter 32. One of the cables includes a fluid tube 60 that extends between the cartridge 36 and the balloon catheter 32. The fluid tube 60 includes a fluid tube connector 62 that may be coupled to the fluid source 38 (FIG. 1). The cartridge 36 is adapted to be coupled with a cartridge connection port provided on a front face of the system controller 34 (FIG. 1).

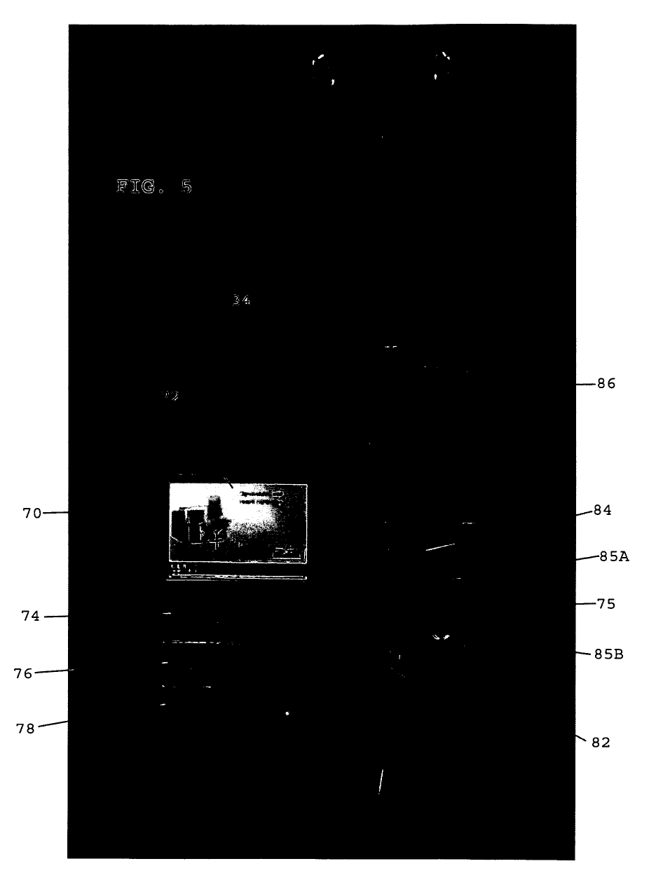

Referring to FIG. 4, the cartridge 36 has a front face 64 including electrical connection pads 66, and a pressure port connector 68. The cartridge 36 also includes the fluid tube 60 coupled with an upper end thereof. The front face 64 of the cartridge 36 also includes a pair of connection posts 69A, 69B projecting therefrom that enhance the stability of the connection between the cartridge and the cartridge connection port of the system controller.

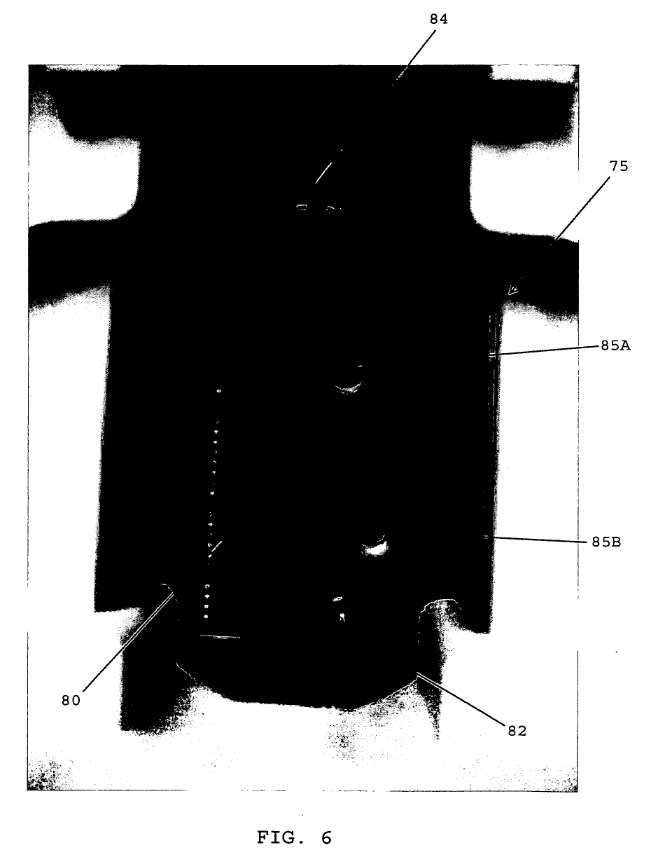

Referring to FIG. 5, the system controller 34 preferably controls operation of the balloon catheter during an endometrial ablation procedure. As such, the system controller preferably has one or more endometrial ablation procedures or subroutines programmed therein. In one embodiment, the system controller has one or more subroutines for automatically priming the balloon catheter with a fluid. The front face 70 of the system controller 34 desirably includes one or more visual displays for monitoring priming of the balloon catheter, the pressure of the fluid inside the inflatable balloon, the temperature level of the fluid inside the inflatable balloon, and the time remaining in a procedure. The one or more visual displays may also provide instructions to an operator and/or enable an operator to track the status of an endometrial ablation procedure.

The system controller is adapted to regulate and control the heat applied to the fluid in the inflatable balloon by modulating the electrical voltage or current to the heater assembly or other power source for the heating assembly. The system controller may include a temperature controller which uses temperature sensors such as thermocouples or thermistors for feedback control. The temperature may be controlled to a predetermined level or to a level selected by an operator. The system controller further controls the operating time for which heat is applied to the fluid in the inflatable balloon and monitors the pressure of the fluid in the inflatable balloon. The system controller also initiates and terminates the operation of the rotary drive mechanism which initiates and terminates the rotation of the impeller drive shaft and the impeller. The system controller may incorporate one or more of the features disclosed in commonly assigned U.S. Pat. Nos. 4,949,718 and 5,800,493, the disclosures of which are hereby incorporated by reference herein in their entirety. Any of the above operations may be performed automatically by the system controller, or the system controller may provide instructions while an operator manually performs any of the above-described operations.

FIG. 5 shows a front face 70 of the system controller 34 including a visual display screen 72 for displaying instructions and/or information related to conducting an endometrial ablation procedure. The front face 70 also includes a first section 74 for displaying information related to the pressure level of the fluid inside the inflatable balloon, a second section 76 for providing information related to the temperature level of the fluid inside the balloon and/or the temperature of a heater inside the balloon, and a third section 78 for displaying information related to the length of an endometrial ablation procedure and/or the time remaining in the procedure. The system controller 34 also includes the cartridge connection port 75 including an electrical connector port 80 adapted to receive the electrical connection pads 66 (FIG. 4) on the front face 64 of the cartridge 36. The cartridge connection port 75 also includes a controller pressure port 82 adapted to receive the pressure port connector 68 on the cartridge 36. The system controller 34 also includes a peristaltic pump 84 that is adapted to engage a portion of the fluid tube 60 provided at an upper end of the cartridge 36 for forcing the fluid into the balloon. The peristaltic pump may be operated in a first direction for introducing fluid into the balloon and a second direction for extracting fluid from the balloon.

The cartridge connection port 75 also preferably includes connection post openings 85A, 85B adapted to receive the connection posts 69A, 69B projecting from the front face of the cartridge. The system controller 34 also preferably includes a cartridge connection port cover 86 movable between an open configuration shown in FIG. 5 and a closed configuration shown in FIG. 2. In the open configuration shown in FIG. 5, the cover 86 is in an up position for exposing the electrical connector port 80, the controller pressure port 82, and the peristaltic pump 84. When the cover 86 is in the open configuration, the front face 64 of the cartridge 36 may be connected with the connection ports of the cartridge connection port 75. In the closed configuration shown in FIG. 2, the cover 86 covers and protects the connection ports of the system controller from exposure and/or contamination.

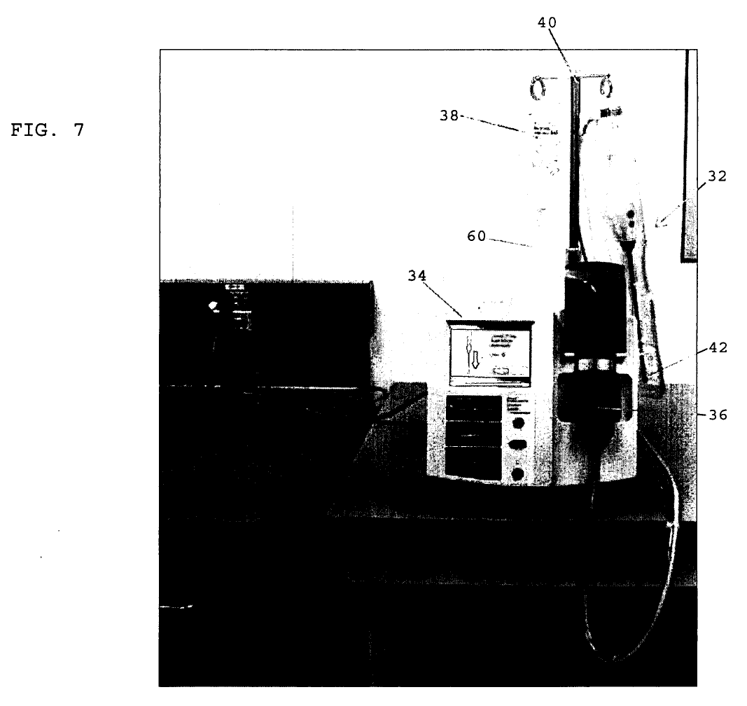

Referring FIG. 6, in one embodiment, the cartridge connection port 75 includes electrical connector port 80 adapted to receive the electrical connectors on the front face of the cartridge. The cartridge connection port 75 also includes controller pressure port 82 adapted to receive the pressure port connector on the front face of the cartridge. The peristaltic pump 84 is preferably located at an upper end of the cartridge connection port 75 so as to engage the fluid tube provided at the upper end of the cartridge. The cartridge connection port 75 also includes attachment port posts 85A, 85B adapted to receive the connection posts 69A, 69B projecting from the front face of the cartridge.

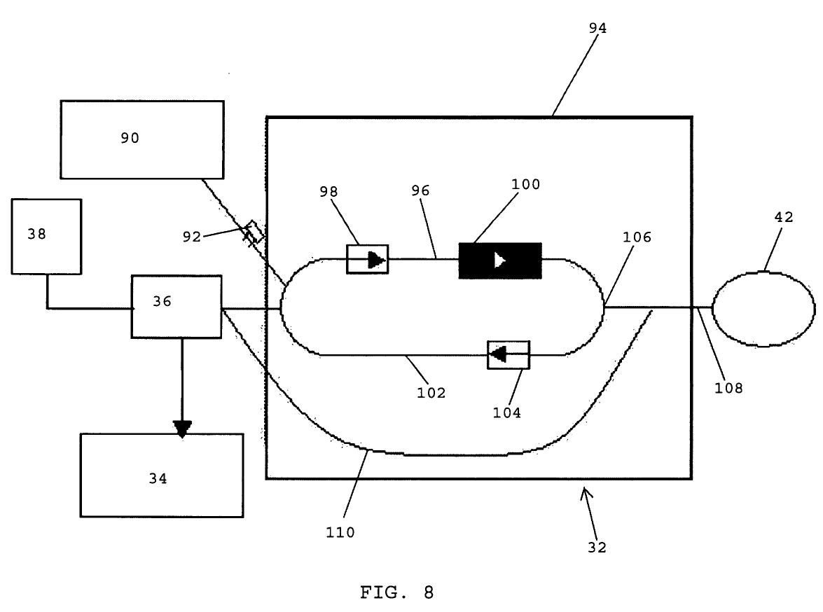

FIG. 7 shows the cartridge 36 coupled with the connection ports of the system controller 34. In one embodiment, during priming of the system, the balloon catheter 32 is held in a downward orientation by the fluid source holder 40 whereby the inflatable balloon 42 at the distal end defines the lowest part of the balloon catheter. The fluid source 38, held by the fluid source holder 40, supplies the fluid to the balloon catheter 32 through the fluid tube 60. The fluid source 38 is preferably in fluid communication with the balloon catheter 38 via the fluid tube 60. The fluid tube 60 is preferably in contact with the peristaltic pump (not shown) shown and described above.

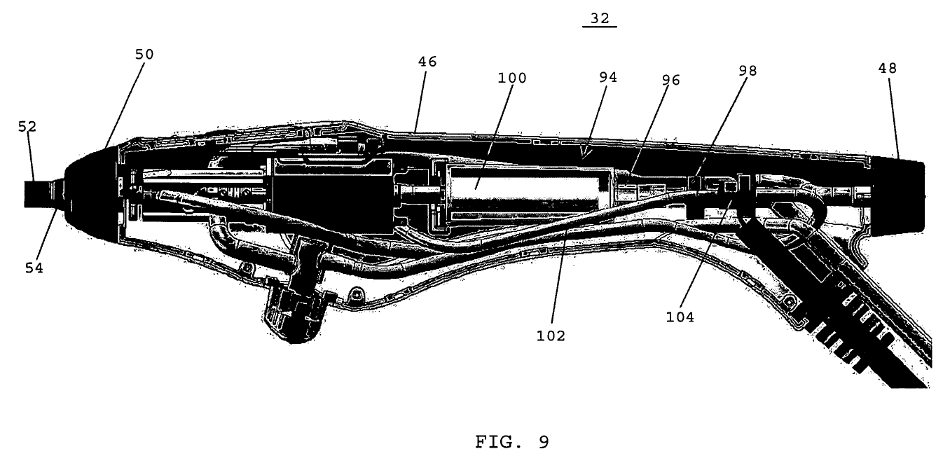

FIG. 8 shows a schematic view of a degassing system for removing gas or air from the fluid introduced into the balloon, in accordance with one embodiment of the present invention. The system includes balloon catheter 32 having an inflatable balloon 42 located at a distal end thereof, and a controller 34 that is coupled with the balloon catheter 32 via cartridge 36. A fluid source 38 is coupled with the balloon catheter 32 via the cartridge 36. The fluid tube 60 for providing fluid to the balloon catheter 32 is in contact with a pump (not shown) provided on the system controller 34. The system 30 includes a manual mode fill port 90 in communication with the balloon catheter 32, and a trumpet valve 92 for controlling introduction of or removal of the fluid from the balloon catheter 32 through the manual mode fill port 90.

The balloon catheter 32 includes the above-mentioned de-grassing system 94. In one embodiment, the degassing system is contained within the handle of the balloon catheter 32. The degassing system 94 includes a fluid insertion path 96 extending between the proximal end of the balloon catheter handle and the distal end of the balloon catheter handle. The fluid insertion path is utilized for introducing fluid into the inflatable balloon 42. The fluid insertion path 96 includes a first check valve 98 and an air filter 100 located downstream of the first check valve 98. The degassing system 94 also includes a fluid extraction path 102 for extracting fluid from the inflatable balloon 42. The fluid extraction path 102 includes a second check valve 104 that enables fluid to be extracted from the inflatable balloon 42. The first check valve 98 is designed to allow fluid to move in only one direction, namely in a distal direction toward the inflatable balloon 42 located at the distal end of the balloon catheter. The second check valve 104 is adapted to allow fluid to move in only one direction, namely in a proximal direction toward the proximal end of the balloon catheter as the fluid is extracted from the inflatable balloon 42. In preferred embodiments, the fluid insertion and extraction paths 96, 102 allow fluid to flow in opposite directions relative to one another. The fluid insertion path 96 and the fluid extraction path 102 intersect one another at a Y connector 106 located downstream of the air filter 100. The degassing system 94 also includes a fluid cannula 108 that enables the fluid to be passed into out of the inflatable balloon 42 through a vent hole located inside the balloon 42.

The system 30 also preferably includes a pressure line 110 that is in communication with the system controller 34 via the cartridge 36. The pressure line 110 enables the system controller 34 to continuously monitor and control the pressure of the fluid inside the inflatable balloon 42. If the pressure within the inflatable balloon 42 is outside of preferred levels, the system controller 34 will automatically adjust the volume of fluid and/or the pressure level of the fluid within the inflatable balloon 42 so as to modify the pressure to a more preferred level.

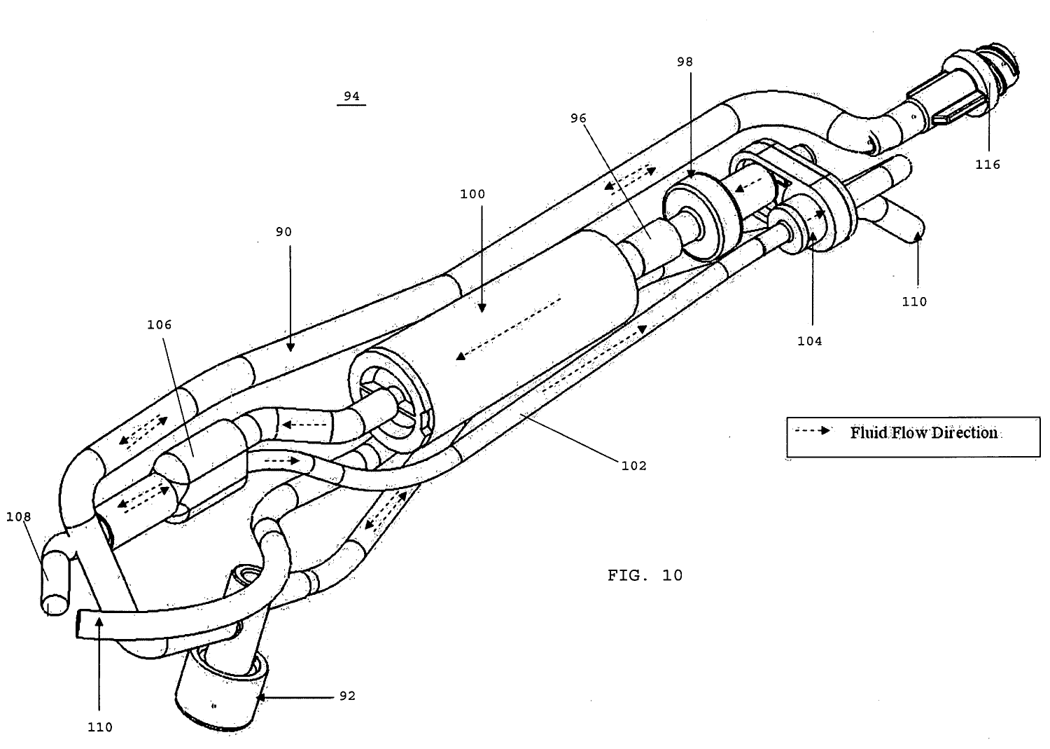

FIG. 9 shows a cross-sectional view of the handle 46 of the balloon catheter 32. The handle 46 has a proximal end 48 and a distal end 50. The proximal end 54 of the cannula 52 is connected with the distal end 50 of the handle 46. In one embodiment, the handle 46 includes an internal compartment that is adapted to receive the degassing assembly 94 shown and described above in FIG. 8. The degassing assembly 94 includes the fluid insertion path 96 having the first check valve 98 and the air filter 100, and the fluid extraction path 102 having the second check valve 104 that allows fluid to flow toward the proximal end 48 of the handle 46.

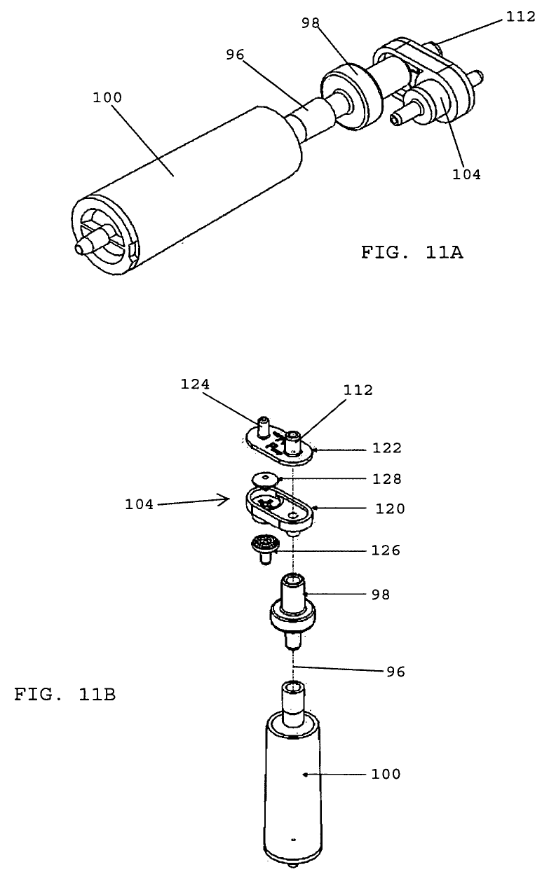

FIG. 10 shows a perspective view of the degassing assembly 94, in accordance with one embodiment of the present invention. The degassing assembly 94 includes the fluid insertion path 96 having the first check valve 98 and the air filter 100 located downstream from the first check valve 98. The first check valve 98 enables the fluid to flow distally in the direction indicated by the fluid flow arrows for filling the inflatable balloon at the distal end of the balloon catheter. The fluid insertion path 96 has a proximal end including a first inlet port 112 that is preferably coupled with the fluid tube 60 (FIG. 3) in communication with the fluid source. The degassing assembly 94 includes Y connector 106 that is in communication with the fluid cannula 108 disposed inside the inflatable balloon. When it is desirable to introduce fluid into the inflatable balloon, the fluid passes into the fluid-fill inlet port 112, through the first check valve 98, through the air filter 100 for removing any gas in the fluid, through the Y connector 106, and is discharged from the distal end of the fluid cannula 108. In one embodiment, the distal end of the fluid cannula 108 is in communication with the vent hole provided inside the inflatable balloon.

The degassing assembly also includes the fluid extraction path 102 having the second check valve 104 in communication therewith. The fluid extraction path 102 has a distal end in communication with the Y connector 106. When it is desirable to extract fluid or gas from the inflatable balloon, the fluid or gas is drawn through the fluid cannula 108, toward the Y connector 106, in a proximal direction through the fluid extraction path 102, and through the second check valve 104.

In one embodiment, the system also includes pressure line 110 that is in communication with the fluid and the system controller (not shown) for monitoring and/or controlling the pressure level of the fluid within the inflatable balloon. The system also preferably includes the manual mode fluid path 90 that enables fluid to be manually introduced into and extracted from the inflatable balloon. The manual mode fluid path 90 has a proximal end including a syringe attachment 116 adapted to be coupled with a distal end of a syringe. The manual mode fluid path 90 includes a trumpet valve 92 in communication therewith that may be engaged (e.g. depressed) for opening the manual mode fluid line 90 so as to enable fluid to be manually introduced into and extracted from the balloon.

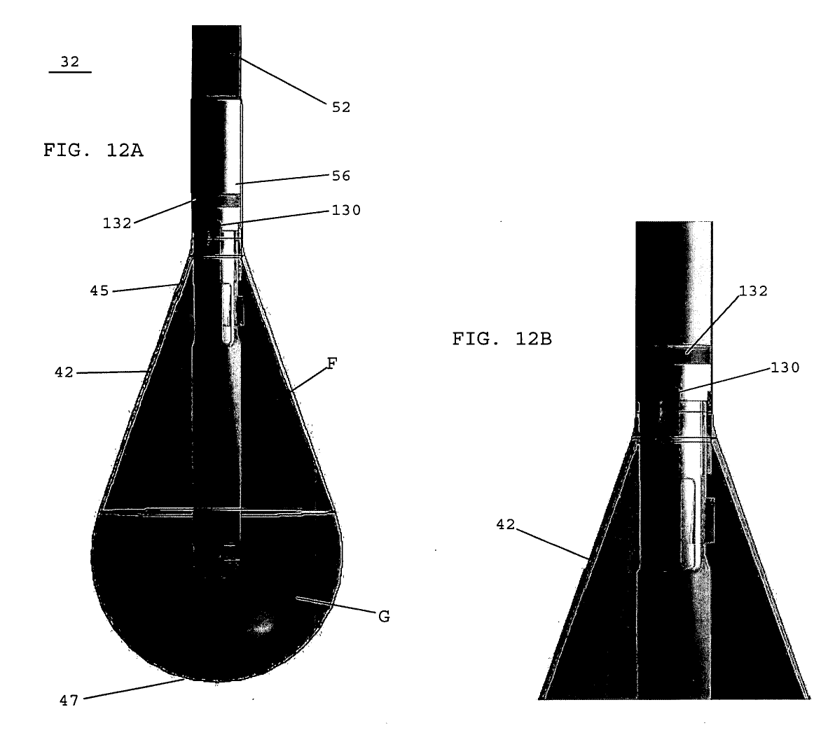

Referring to FIG. 11A, in one embodiment, the degassing system includes the first check valve 98 and the air filter 100 located downstream of the first check valve 98. The first check valve 98 and the air filter 100 are in communication with one another via the fluid insertion path 96 extending therebetween. The proximal end of the fluid insertion path 96 includes a first inlet coupler 112 adapted to be coupled to the fluid tube (not shown). The degassing system also includes the second check valve 104 that is part of the fluid extraction path described above in FIG. 8.

FIG. 11B shows an exploded view of the components described above in conjunction with FIG. 11A. The system includes first check valve 98 that is adapted to be in communication with air filter 100 along the fluid insertion path 96. The degassing system includes a valve platform 120 and a valve cap 122 received within a recess formed atop the valve platform 120. The valve cap 122 includes the first inlet port 112 for introducing the fluid into the fluid insertion path 96. The valve cap 122 includes a second discharge port 124 used to extract fluid from the inflatable balloon. The degassing system includes the second check valve 104 disposed in contact with the valve platform 120. The second check valve 104 includes second check valve cap 126 coupled with an underside of the valve platform 120 and an umbrella valve 128 provided in the recess atop the valve platform 120. The second check valve 104 enables fluid to pass in only one direction, namely toward the proximal end of the balloon catheter so as to be discharged through the second discharge port 124.

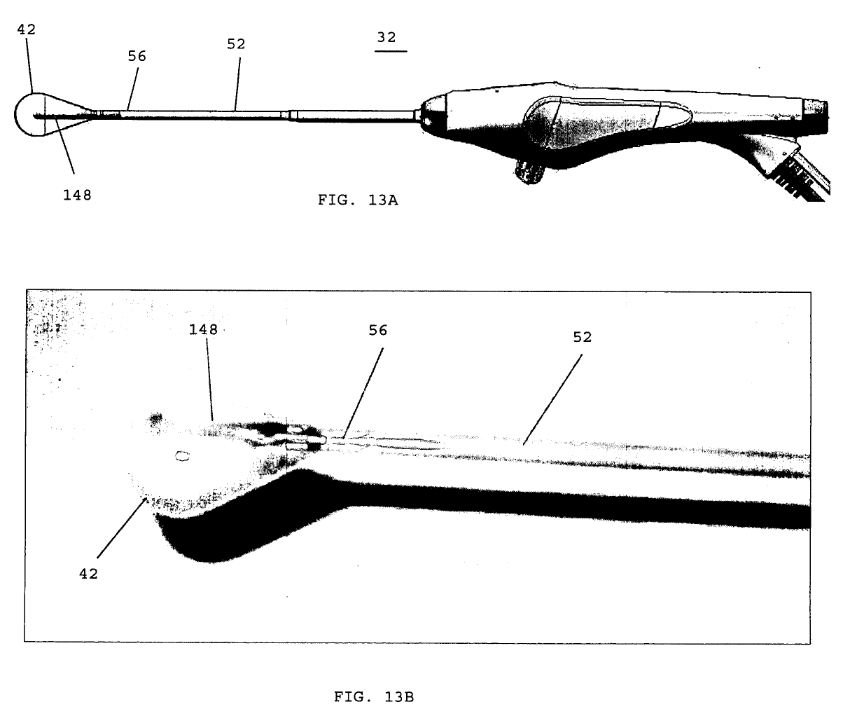

FIG. 12A shows the balloon catheter 32 during a fluid priming operation whereby the balloon catheter 32 and the inflatable balloon 42 located at the distal end of the balloon catheter are pointed in a downward direction. As the balloon 42 is pointed downward, the proximal end 45 of the balloon 42 defines the highest section of the balloon and any gas G remaining inside the balloon moves to this highest section, which is adjacent vent hole 130 at the distal end 56 of the cannula 52. In the downward position of FIG. 12A, the fluid F inside the balloon flows to the distal end 47 of the balloon 42. In one embodiment, a syringe may be coupled with the balloon catheter for priming the balloon. However, in preferred embodiments, the fluid may be introduced into the balloon catheter automatically using the system controller, the peristaltic pump and the fluid tube shown and described above.

Referring to FIGS. 12A and 12B, the distal end 56 of the cannula 52 includes the vent hole 130 disposed inside of the inflatable balloon 42. The vent hole 130 is in communication with the fluid cannula 108 shown and described above in FIG. 10. The fluid is preferably introduced into the inflatable balloon 42 through the vent hole 130. The fluid F and the gas G may be extracted from the inflatable balloon 42 through the vent hole 130. With the balloon in the downward orientation shown in FIGS. 12A and 12B, any gas G remaining inside the inflatable balloon 42 will flow to the highest point of the balloon, which is located adjacent the vent hole 130. As a result, any gas G remaining in the inflatable balloon 42 may be extracted from the balloon by drawing the gas through the vent hole 130, the fluid extraction path 102, and the second check valve 104 (FIG. 10). A series of cyclical steps may be repeated whereby fluid is introduced into the balloon 42 through the fluid insertion path and then extracted from the balloon through the fluid extraction path 102. As the steps are repeated, and as fluid is introduced through the fluid insertion path 96, any air or gas in the fluid is removed by the air filter 100. If any gas G remains inside the balloon 42, the gas is removed as the fluid and/or gas is extracted via the fluid extraction path. When fluid is being extracted, the first check valve is closed for preventing any fluid and air from passing through the fluid insertion path 96. Moreover, fluid introduced into the balloon must pass through the air filter, which also removes any gas and/or air present in the fluid. As fluid is extracted, it cannot move proximally through the air filter 100 but can only be removed through the fluid extraction path in communication with the second check valve 104.

In one embodiment, the distal end of the balloon catheter 32 includes a silicon sleeve 132 and a suture for securing the inflatable balloon 42 to the distal end 56 of the cannula 52. The distal end of the cannula also includes the vent hole 130 in communication with the fluid insertion path 96 (not shown). The proximal end 45 of the balloon 42 preferably covers the vent hole 130 so that the vent hole is located inside the balloon.

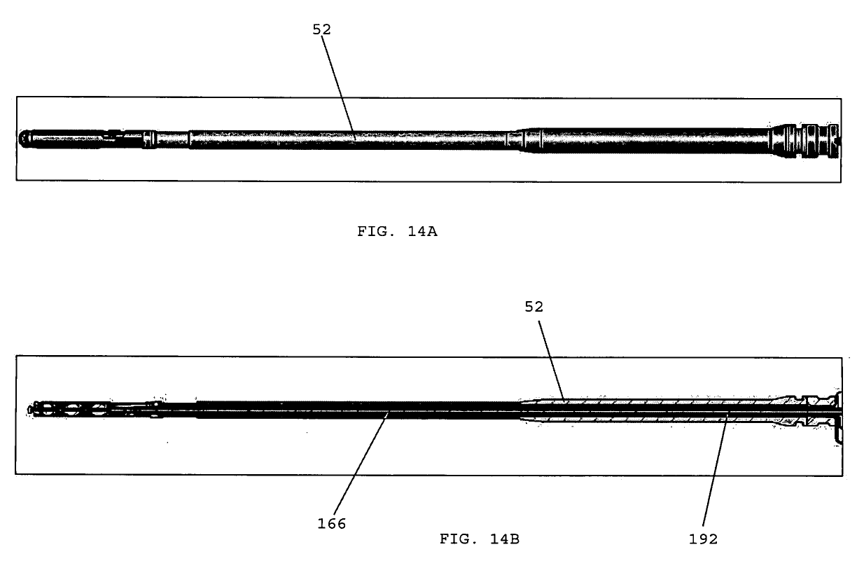

Referring to FIGS. 13A and 13B, in one embodiment of the present invention, a distal end of the balloon catheter 32 includes the distal end 56 of the cannula 52 and the inflatable balloon 42 connected to the cannula. The balloon catheter also includes a heating assembly 148 extending from the distal end 44 of the cannula. The heating assembly 148 is disposed inside the balloon for heating the fluid introduced into the balloon. The balloon catheter also includes a rotatable impeller (not shown) disposed inside the heating assembly for drawing fluid into engagement with the heating assembly, heating the fluid, and circulating the fluid throughout the inside of the balloon to provide for uniform heating of the outer surface of the balloon.

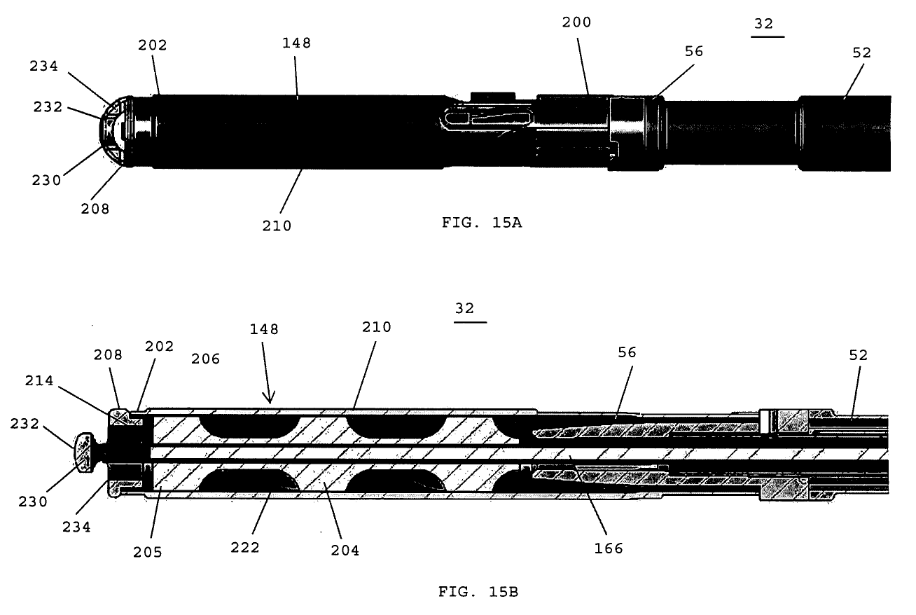

Referring to FIG. 14A, in one embodiment of the present invention, the cannula 52 is preferably an elongated tube that may be flexible and/or semi-rigid. In one embodiment, the cannula is made of silicone with a metal tube provided at the center. In other embodiments, the cannula may be made of materials such as acrylonitrile-butadiene-styrene (ABS), polyvinyl-chloride (PVC), or polyurethane. The cannula is preferably insertable into the uterus, while providing support necessary for manipulating the position of the inflatable balloon within the uterus. The cannula 52 desirably has a sufficient length from the inflatable balloon to the balloon catheter handle to extend through a patient's vaginal canal, the cervix and into the uterus. Placement of the device may be aided by virtue of scale gradations provided on the outer surface of the cannula to indicate the depth of insertion of the inflatable balloon into the uterine cavity.

In one embodiment of the present invention, the cannula 52 desirably has a lumen 192 adapted to receive a fluid, an impeller drive shaft for rotating the impeller, and electrical leads for the heater assembly, thermistors, the impeller and/or any other components required to be interconnected with the system controller. The lumen 192 preferably extends along the length of the cannula 52 between the balloon catheter handle and the distal end of the cannula. The lumen may be arranged in any configuration required while maintaining the structural integrity of the cannula shaft. The cross-sectional shape of the lumens may be annular, hemispherical, or any other shape suitably required for performance of the device.

Referring to FIG. 14B, in one embodiment, an impeller drive shaft 166 is positioned centrally within the lumen 192 so that contact along the length of the drive shaft with the wall of the lumen is minimized for reducing friction. The proximal end of the drive shaft 166 is desirably in communication with the system controller. The distal end of the lumen 192 is in communication with the inside of the heating assembly. Electrical leads interconnecting the system controller with the heating assembly, the impeller and/or thermocouples may also extend through the lumen 192. In one embodiment, the space between the inner diameter of the heating tube and the drive shaft for the impeller is used exclusively for introducing fluid into and removing fluid from the inflatable balloon. In one embodiment, the electrical leads for the heater and the thermistor are located outside the heating tube and may be embedded in silicone.

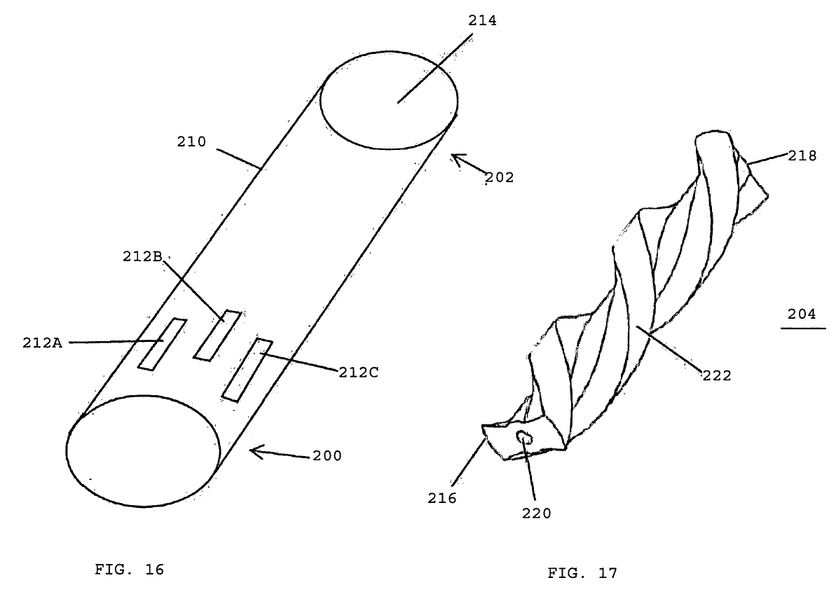

Referring to FIGS. 15A and 15B, in one embodiment of the present invention, the heating assembly 148 projects from the distal end 56 of the cannula 52. In FIGS. 15A and 15B, the inflatable balloon secured to the distal end of the balloon catheter has been removed so that the heating assembly and the impeller may be clearly seen. The heating assembly 148 has a proximal end 200 coupled with the distal end 56 of the cannula 52 and a distal end 202 remote therefrom. The heating assembly 148 preferably includes an elongated tubular member extending between the proximal end 200 and the distal end 202 thereof. A heating film may overlie the outer surface of the elongated tubular member for generating heat. The elongated heating tube may incorporate one or more of the medical heater technologies sold under the trademark MICROPEN by MicroPen Technologies of Honeoye Falls, N.Y. In one embodiment, the heating element may be made of any thermally conductive material. In one embodiment, the heating element is preferably a metal tube such as a stainless steel metal tube. A conductive film may be provided over the outer surface of the metal tube. The conductive film is preferably adapted to generate heat that is transferred to fluid passing through the heating assembly. The conductive film may be a conductive ink that is printed over the outer surface of the tube. The conductive ink may be printed in a pattern. The conductive film may also be provided over an inner surface of the heating tube.

In one embodiment, the heating assembly may incorporate one or more of the fluid heating elements sold by Watlow Electric Manufacturing Company of St. Louis, Mo., including the heater technology disclosed in U.S. Pat. No. 6,944,394, the disclosure of which is hereby incorporated by reference herein. The balloon catheter 32 desirably includes a rotatable impeller 204 that is disposed within the tubular heating assembly 148. In one embodiment, the impeller has a length that lies completely within the extent of the heating assembly. As such, the heating assembly may entirely encompass the impeller.

In one highly preferred embodiment, the distal end 202 of the heating assembly is distal to the distal end 205 of the impeller 204. The impeller 204 is connected to an impeller drive rod 166 that rotates the impeller 204 inside the heating assembly 148. The impeller drive rod 166 is preferably about 0.5 to 1.0 millimeters in diameter, and desirably has some flexibility. The impeller drive rod may be made of stainless steel or spring steel. The impeller drive rod desirably extends the entire length of the balloon catheter from the distal end of the balloon to the balloon catheter handle. In other embodiments, a co-axially wound cable is also suitable. A distal end 206 of the impeller drive rod 166 is covered by a protective cap 208 that is adapted to prevent the inflatable balloon from being damaged by the distal end 206 of the drive rod or the distal end of the heating assembly 148.

Referring to FIGS. 15A and 15B, the heater assembly 148 includes an elongated tube having a proximal end 200, a distal end 202, and a tubular outer wall 210 extending therebetween. The balloon catheter include the rotatable impeller 204 disposed within the heater assembly. The rotatable impeller 204 is rotated by the impeller drive shaft 166 that extends through the cannula 52 and the heater assembly 148. The distal end 206 of the drive shaft 166 is preferably covered by the protective cap 208. In one embodiment, the protective cap 208 includes a central hub 230 having a curved or a convexly curved distal surface 232 and an opening 234 that surrounds the central hub 230. The opening 234 enables the fluid discharged from the discharge opening 214 of the heater to pass therethrough.

In one embodiment, when the impeller rotates, pressure gradients formed by the helical threads 222 on the impeller 204 draw fluid into the heating assembly through the fluid inlet ports. The rotating impeller also causes fluid to exit the distal end of the heating assembly through the fluid outlet and through the protective cap. Thus, a circulation path of fluid within the balloon, proximal to distal to proximal, is developed. The circulation path of the fluid preferably circulates the fluid throughout the entire balloon and preferably minimizes temperature gradients at the outer surface of the inflated balloon.

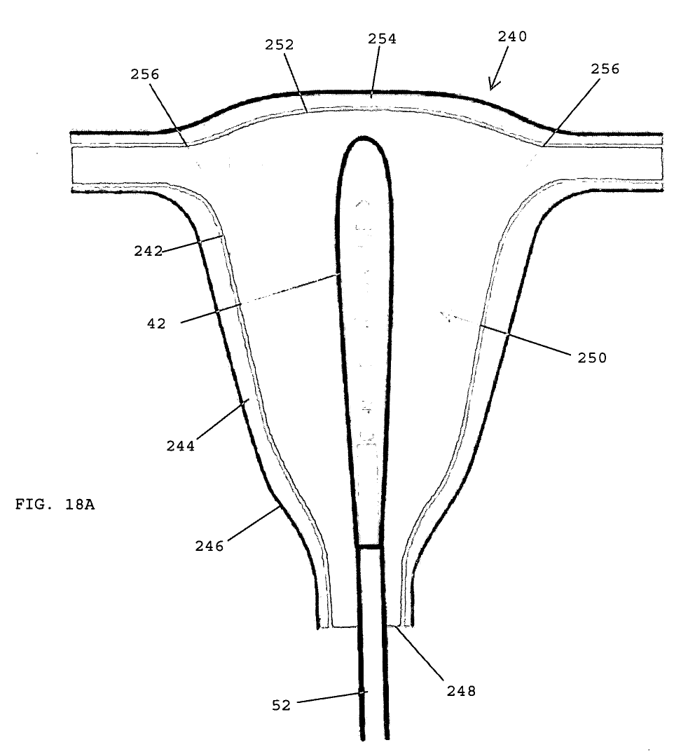

FIG. 16 shows a perspective view of a section of the heating assembly 148, in accordance with one embodiment of the present invention. The heating assembly 148 includes an elongated tubular member having a proximal end 200 and a distal end 202. The heating assembly 148 includes an outer wall 210 having an outer surface. One or more films adapted to generate heat may overlie the outer surface of the outer wall. The heating assembly 148 includes a series of fluid inlets 212A, 212B, 212C that enable fluid to pass from outside the heating assembly to inside the heating assembly. As the fluid passes through the fluid inlets, the fluid preferably contacts the heating assembly for heating the fluid. The heating assembly 148 also includes a fluid outlet 214 provided at a distal end 202 thereof for discharging the heated fluid from the distal end of the heating assembly, and for efficiently circulating the fluid throughout the balloon. The total area of the fluid inlets 212A-212C is preferably at least equal to the total area of the fluid outlet 214. The fluid inlets are preferably located at the proximal end 200 of the heater tube 148 so as to increase contact between the fluid and the heater tube as the fluid flows along the length of the heater tube.

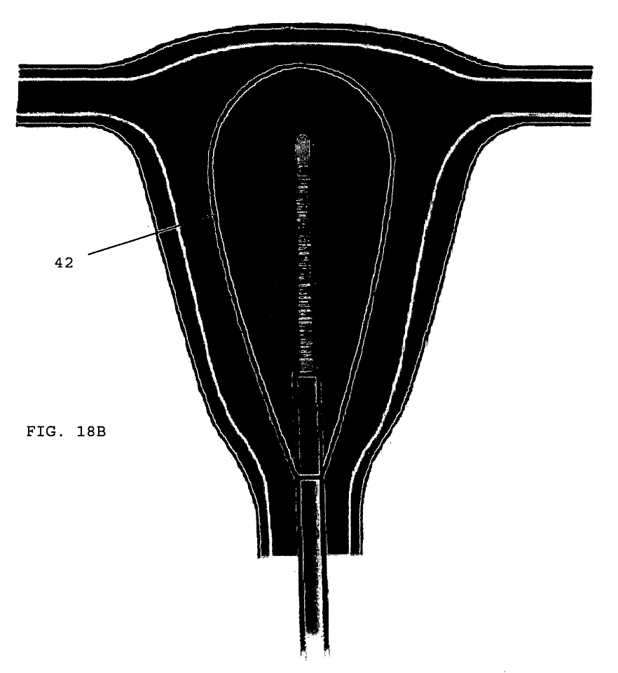

FIG. 17 shows a rotatable impeller 204 in accordance with one embodiment of the present invention. The impeller 204 includes a proximal end 216, a distal end 218 and a drive shaft lumen 220 that extends between the proximal and distal ends. The impeller 204 preferably includes helically wound threads 222. The impeller may have a single thread or multiple threads. In one preferred embodiment, the impeller is a double thread impeller that extends between the proximal and distal ends thereof. In other preferred embodiments, the impeller may include blades or fins for circulating fluid. As the impeller 204 is rotated by the drive rod 166 (FIG. 15B), the helical screw threads 222 circulate the fluid inside the balloon. Referring to FIGS. 16 and 17, in one embodiment, the rotating impeller 204 draws fluid into the heater 148 through the fluid inlets 212A-212C, and discharges the fluid from the heater through the fluid outlet 214. The impeller may be made of polymer materials such as polycarbonate (PC), latex strips, polyethylene (PE), polyethylenetherapthalate (PET) or other suitable materials such as metals and alloys.

The elongated heating tube preferably has an inner diameter that is slightly larger than the outer diameter of the impeller. The action of the rotating impeller causes circulation of the fluid through the heating tube and within the balloon. In one embodiment, the heating assembly includes a fluid thermister for monitoring the temperature of the fluid inside the balloon. The heating assembly 148 also preferably includes a heater thermistor for monitoring the temperature of the heater.

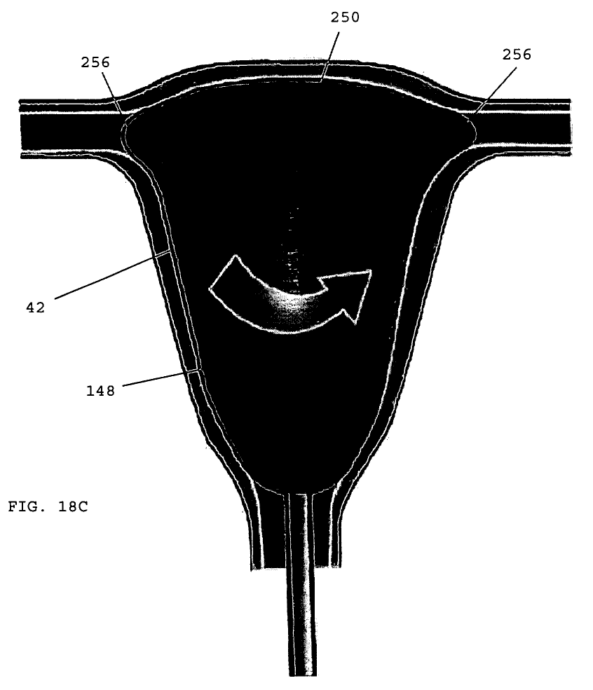

FIGS. 18A-18C show a balloon catheter system during an endometrial ablation procedure, in accordance with one embodiment of the present invention. Referring to FIG. 18A, the inflatable balloon 42 at the distal end of the cannula 52 is aligned for insertion into a uterus 240. The uterus 240 has three basic layers, i.e., the endometrium 242, the myometrium 244 and the outer layer or serosa 246. The balloon catheter is inserted into the uterus through the cervix 248, and is advanced into the uterine cavity 250 until it reaches the distal wall 252 proximate the fundus 254. The inflatable balloon 42 is adapted to conform to the shape of the uterine cavity 250 so as to provide for effective heat transfer from the heating assembly to the endometrium 242. When the balloon is inflated with a fluid, the distal portions of the balloon preferably extend into each cornu 256 of the uterus 240.

Referring to FIG. 18B, fluid is preferably introduced into the inflatable balloon 42. In one embodiment, the fluid may be introduced manually using a syringe. In one embodiment, the fluid may be automatically introduced into the balloon 42 via a peristaltic pump, such as a peristaltic pump provided on the system controller. As the fluid is introduced into the balloon 42, the internal pressure of the fluid in the balloon is continuously monitored to insure that the fluid pressure inside the balloon does not exceed safe pressure levels. Referring to FIG. 18C, a sufficient volume of fluid is preferably introduced into the inflatable balloon 42 until the outer surface of the balloon conforms to the walls of the uterine cavity 250. As the balloon is filled, the pressure level of the fluid is continuously monitored to insure safe pressure levels are maintained within the balloon.

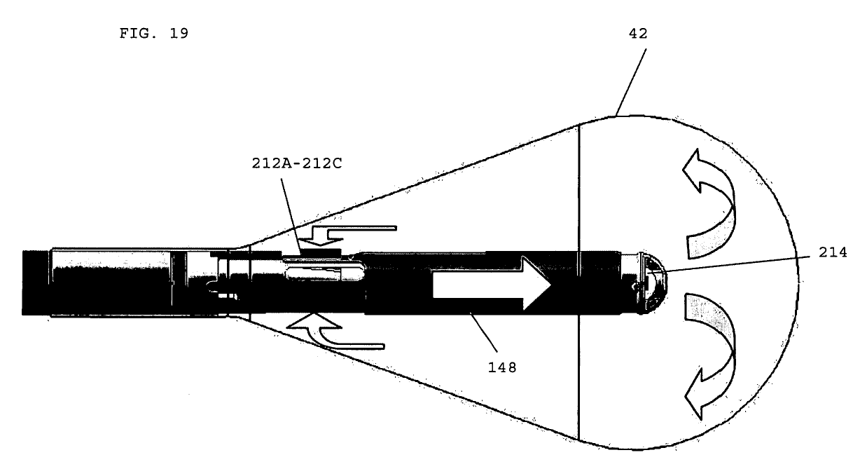

Referring to FIGS. 18C and 19, after a sufficient volume of fluid has been introduced into the balloon 42, the system controller preferably activates the heater 148 for heating the fluid inside the balloon 42. In one embodiment, the heater temperature set point is preferably set to a temperature of about 81° C. to achieve a preferred balloon surface temperature. In other embodiments, the heater temperature set point is set to a temperature that is sufficient for successfully completing endometrial ablation procedures. Thus, various heater temperature set points may be used and still fall within the scope of the present invention. The impeller 204 (FIG. 15B) inside the heater assembly 148 is rotated for drawing the fluid into the fluid inlets 212A-212C of the heating assembly and discharging the heated fluid from the fluid outlet 214 at the distal end of the heating assembly 148. FIG. 19 shows the circulation path of the fluid inside the balloon. The fluid is preferably discharged from the fluid outlet 214 at the distal end of the elongated heating tube and circulated throughout the balloon, including the portions of the balloon in the vicinity of the cornua 256 (FIG. 18C). The fluid is drawn into the fluid inlets 212A-212C and directed toward the fluid outlet by the rotating impeller. The fluid is then directed through the inside of the heating tube toward the distal end of the heating tube. Heat is transferred from the heating assembly to the fluid as the fluid passes closely by the inner surface of the heating assembly.

In one embodiment, heat is applied to the fluid by applying electric voltage to the heating assembly, and the impeller is rotated for circulating the fluid throughout the balloon. The rotation of the impeller preferably continues for the duration of the heat therapy. At the end of the procedure, the heating assembly is deactivated. After the power to the heating assembly is turned off, it is preferable to maintain the rotation of the impeller until the fluid is drained from the balloon.

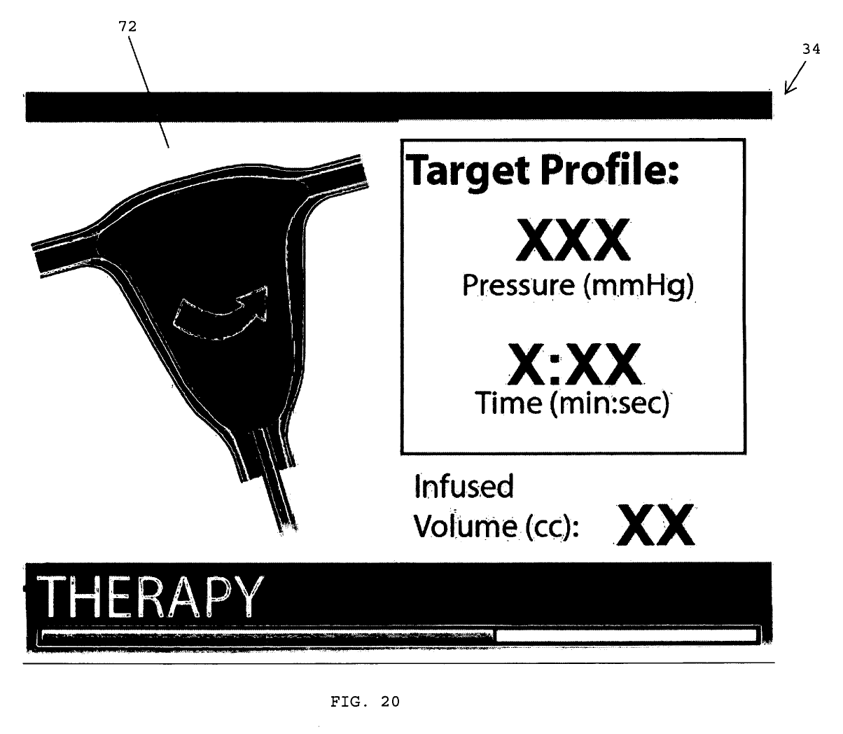

Referring to FIG. 20, during the endometrial ablation procedure, an operator may continuously monitor the visual display screen 72 provided on the front face of the system controller 34 to insure that the procedure is advancing within proper parameters. As noted above, an operator will monitor the priming of the balloon with fluid, the pressure of the fluid inside the balloon, the temperature of the fluid inside the balloon, the status of the procedure, and the amount of time remaining in the procedure. The operator may also monitor the visual display screen to receive instructions and/or observe the status of the procedure. In one embodiment, any one of the above steps may be automatically controlled by the system controller.

After the endometrial ablation procedure is completed, the fluid inside the balloon is withdrawn from the balloon through the cannula. The fluid is preferably cooled inside the balloon before it is withdrawn through the cannula. As the fluid is withdrawn, the inflatable balloon collapses. After all of the fluid has been withdrawn from the inflatable balloon, the inflatable balloon returns to its initial collapsed position. The distal end of the balloon catheter may then be removed from the uterine cavity.

The headings used herein are for organizational purposes only and are not meant to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

Although particular embodiments of the present invention have been illustrated and described herein, various modifications may be made without departing from the spirit and scope of the invention, and other and further embodiments of the invention may be devised without departing from the basic scope thereof. For example, it is contemplated that the degassing system disclosed herein may be incorporated into any type of medical device and still fall within the scope of the present invention. Accordingly, the present disclosure is not intended to limit the scope of the invention, which is defined by the appended claims.

What is claimed is:

1. A system for treating uterine disorders comprising:
a catheter including a cannula having a proximal end and a distal end; and
a degassing system in communication with the distal end of said catheter, said degassing system including a fluid insertion path having a first check valve and a gas filter, and a fluid extraction path separate from the fluid insertion path having a second check valve.

2. The system as claimed in claim 1, further comprising an inflatable balloon secured to the distal end of said cannula, wherein said degassing system is in communication with said inflatable balloon.

3. The system as claimed in claim 1, further comprising a heating assembly disposed inside said inflatable balloon.

4. The system as claimed in claim 2, wherein said first check valve allows fluid to flow toward said inflatable balloon and said second check valve allows fluid to flow away from said inflatable balloon.

5. The system as claimed in claim 4, further comprising a system controller in communication with said balloon catheter for controlling operation of said balloon catheter, wherein said system controller includes a priming subroutine for filling said inflatable balloon with a fluid having at least one fluid insertion phase and at least one fluid extraction phase.

6. The system as claimed in claim 5, wherein said first check valve is open and said second check valve is closed during the at least one fluid insertion phase.

7. The system as claimed in claim 5, wherein said first check valve is closed and said second check valve is open during the at least one fluid extraction phase.

8. The system as claimed in claim 3, wherein said gas filter is located distally from said first check valve and is adapted to remove gas present in fluid flowing through the fluid insertion path.

9. The system as claimed in claim 8, wherein the distal end of said cannula includes a vent hole disposed inside said inflatable balloon adjacent a proximal end of said inflatable balloon, and wherein the fluid insertion and fluid extraction paths are in communication with said vent hole.

10. A system for treating uterine disorders comprising:
a balloon catheter including a handle, and a cannula extending from a distal end of said handle;
an inflatable balloon secured to a distal end of said cannula;
a heating assembly disposed inside said inflatable balloon;
a fluid agitator disposed inside said inflatable balloon;
a fluid degassing system disposed within said handle and being in communication with said inflatable balloon including
a fluid insertion path having a first check valve and a gas filter, and
a fluid extraction path separate from the fluid insertion path and having a second check valve.

11. The system as claimed in claim 10, wherein said first check valve allows fluid to flow in a distal direction toward said inflatable balloon and said second check valve allows fluid to flow in a proximal direction away from said inflatable balloon.

12. The system as claimed in claim 10, further comprising a system controller in communication with said balloon catheter for controlling operation of said balloon catheter, wherein said system controller includes a priming subroutine for filling said inflatable balloon with a fluid having at least one fluid insertion phase and at least one fluid extraction phase.

13. The system as claimed in claim 12, wherein said first check valve is open and said second check valve is closed during the at least one fluid insertion phase, and wherein said first check valve is closed and said second check valve is open during the at least one fluid extraction phase.

14. The system as claimed in claim 10, wherein said heating assembly comprises an elongated heating tube having a proximal end, a distal end, and an outer wall extending between the proximal and distal ends, at least one fluid inlet extending through said outer wall, and a fluid outlet located at the distal end of said elongated heating tube, and wherein said fluid agitator is a rotatable impeller disposed inside said elongated heating tube for drawing fluid through said at least one fluid inlet and into said elongated heating tube for heating fluid inside said balloon.

15. The system as claimed in claim 14, wherein said at least one fluid inlet is located adjacent the proximal end of said elongated heating tube, and wherein said rotatable impeller is adapted to discharge the fluid through said fluid outlet located at the distal end of said elongated heating tube so as to circulate the fluid throughout said inflatable balloon.

16. A system for treating uterine disorders comprising:
a balloon catheter including a handle, and a cannula extending from a distal end of said handle;
an inflatable balloon secured to a distal end of said cannula and being adapted to receive fluid; and
a fluid degassing system disposed within said handle and being in communication with the inside of said inflatable balloon, said fluid degassing system including a fluid insertion path having a first check valve and a gas filter, and a fluid extraction path separate from the fluid insertion path and having a second check valve.

17. The system as claimed in claim 16 wherein said first check valve allows the fluid to flow in a distal direction toward said inflatable balloon and said second check valve allows the fluid to in a proximal direction away from said inflatable balloon.

18. The system as claimed in claim 17, further comprising a system controller in communication with said balloon catheter for controlling operation of said balloon catheter, wherein said system controller includes a priming subroutine for filling said inflatable balloon with a fluid having at least one fluid insertion phase and at least one fluid extraction phase, and wherein said first check valve is open and said second check valve is closed during the at least one fluid insertion phase, and said first check valve is closed and said second check valve is open during the at least one fluid extraction phase.

19. The system as claimed in claim 16, wherein the distal end of said cannula includes a vent hole and said inflatable balloon has a proximal end that overlies said vent hole so that said vent hole is located adjacent the proximal end of said inflatable balloon, and wherein the fluid insertion and fluid extraction paths are in communication with said vent hole.

20. The system as claimed in claim 18, further comprising:
a heating assembly disposed inside said inflatable balloon for heating the fluid; and
a rotatable impeller disposed inside said inflatable balloon and adjacent said heating assembly for circulating the fluid through the inside of said inflatable balloon, wherein said heating assembly comprises an elongated heating tube having a proximal end, a distal end, and an outer wall extending between the proximal and distal ends, at least one fluid inlet extending through said outer wall of said heating tube, and a fluid outlet located at the distal end of said elongated heating tube, and wherein said rotatable impeller is disposed inside said elongated heating tube for drawing fluid through said at least one fluid inlet and into said elongated heating tube for heating fluid inside said balloon.
